(19) United States
(12) Patent Application Publication
(10) Pub. No.: US 2020/0230438 A1
(43) Pub. Date: Jul. 23, 2020

(54) A couch-mounted stereoscopic surface imaging and biofeedback system

(71) Applicant: Opus Medical Ltd., Eveleigh, NSW (AU)

(72) Inventors: Sean Pollock, Oxford (GB); Paul J. Keall, Greenwich, NSW (AU)

(21) Appl. No.: 16/648,149

(22) PCT Filed: Oct. 11, 2018
(86) PCT No.: PCT/EP2018/077685

§ 371 Date: Mar. 17, 2020

Related U.S. Application Data
(60) Provisional application No. 62/570,817, filed on Oct. 11, 2017.

(51) Int. Cl. A61N 5/10

(52) U.S. Cl. CPC A61N 5/1049; A61N 5/1045; H05H 9/00; A61N 2005/1058; A61N 2005/1087; A61N 2005/1059

(57) ABSTRACT

A patient-guided surface stereoscopic imaging and biofeedback system is provided that includes a patient couch mounting system, an array of at least two imaging sensors, a viewing screen that displays images derived from the imaging sensors to a patient, where the imaging sensors, the viewing screen, and the controller are configured to output to a user 3D surface information of the patient under test, extrapolated 2D patient under test position information, and 1D patient under test position information, where the controller is configured to control the viewing screen to display the images from the imaging sensors, where the viewing screen further displays patient position boundary markers that are configured to overlay the displayed images on the viewing screen to provide biofeedback to a patient under test during radiotherapy treatment.

### FIELD OF THE INVENTION

The present invention relates generally to patient imaging. More particularly, the invention relates to real-time biofeedback to a patient for self-positioning and 3D surface imaging during radiotherapy treatment.

### BACKGROUND OF THE INVENTION

Room-mounted stereoscopic surface imaging solutions for imaging, cancer radiotherapy and other procedures exist. These are expensive, require room modifications for mounting, and have challenges when imaging patients in closed bore imaging and/or radiotherapy systems, e.g. CT, MRI and PET scanners, Halcyon, TomoTherapy. There are also challenges with room-mounted systems where the gantry can obscure the view of the camera system. The increased distance between the cameras and the patients also limits the achievable accuracy and precision of the system.

Furthermore, many patients undergo a battery of exhaustive treatments involving highly restrictive and uncomfortable adjunct equipment to immobilize the patient's position. E.g. 74% of head and neck cancer patients receive radiotherapy, the majority of which require a facemask to pin their head to the treatment couch. The limitations of current radiotherapy treatments require the patient to be completely still during treatment delivery thus necessitating the uncomfortable and claustrophobic immobilization equipment. A technology that adapts to the patient would negate the need for these uncomfortable adjunct immobilization devices in addition to increasing treatment efficiency as current immobilization devices are time-consuming and cumbersome to setup. Facemasks also add build-up material which increases the radiation dose to the patient's skin, increasing the likelihood of toxicities, therefore a further benefit of removing the mask and similar devices is improved safety and outcomes for the patient.

### SUMMARY OF THE INVENTION

To address the needs in the art, a patient-guided surface stereoscopic imaging and biofeedback system that includes a patient couch mounting system, an array of at least two imaging sensors, a viewing screen configured to display images from the imaging sensors, a controller configured to control the imaging sensors and the viewing screen, where the patient couch mounting system is configured to position the imaging sensors for imaging a patient under test on a patient couch from multiple viewing angles, wherein the patient couch mounting system is fixedly attachable to the patient couch, where the viewing screen is disposed in a position that is viewable by the subject under test on the patient couch during the sensor imaging, where the imaging sensors, the viewing screen, and the controller are configured to output to a user 3D surface information of the patient under test, extrapolated 2D patient under test position information, and 1D patient under test position information, where the controller is configured to control the viewing screen to display the images from the imaging sensors, where the viewing screen further displays patient position boundary markers, where the patient position boundary markers are configured to overlay the displayed images on the viewing screen to provide biofeedback to a patient under test during radiotherapy treatment.

According to one aspect of the invention, the biofeedback informs the patient under test of a correct position to adjust to and to maintain.

In another aspect of the invention, the biofeedback system includes a gamified interface, an augmented reality interface, or a gamified interface and an augmented reality interface for visual biofeedback.

In a further aspect of the invention, the imaging sensors can include a camera, an infra-red imager, or an ultrasound imager, where the sensors are configured to operate independently or simultaneously.

According to one aspect of the invention, the imaging sensors are connected to the viewing screen, or separated from the viewing screen.

In yet another aspect of the invention, the imaging sensors are positioned over any region of the patient couch by the patient couch mounting system.

In another aspect of the invention, the imaging sensors and the viewing screen comprise a wireless connectivity, or a wired connectivity.

According to a further aspect of the invention, the patient couch mounting system is detachably mounted to the patient couch.

In one aspect of the invention, the patient-guided surface stereoscopic imaging and biofeedback system is integrated with a gating interface of a cancer therapy system.

In a further aspect of the invention, the patient-guided surface stereoscopic imaging and biofeedback system is compatible with photon therapy, or compatible with proton therapy.

In yet another aspect of the invention, the patient-guided surface stereoscopic imaging and biofeedback system is compatible with the controllable axes of a linear accelerator can include multileaf collimator positions, couch positions, couch angles, collimator angles, or gantry angles.

In a further aspect of the invention, the patient-guided surface stereoscopic imaging and biofeedback system is MRI compatible.

According to one aspect of the invention, the positioning of the imaging sensors or the viewing screen include automated or manual positioning.

In another aspect, the invention further includes collision detection for patient safety.

### BRIEF DESCRIPTION OF THE DRAWINGS

FIG. 1 shows a schematic drawing of the patient-guided stereoscopic surface imaging and biofeedback system, according to one embodiment of the invention.

FIGS. 2A-2B show schematic diagrams of one embodiment of the couch-mounted stereoscopic surface imaging and biofeedback system viewed from (a) the side, and (b) the top, according to one embodiment of the invention.

FIGS. 3A-3C show schematic drawings of the viewing screen showing an augmented reality of the patient positioning with position guidelines and real-time patient positioning, according to one embodiment of the invention.

FIGS. 4A-4F show an example of stereoscopic surface imaging, where 3D surface information (FIG. 4F) is extrapolated from 2D and 1D position information (FIG. 4A-4E), according to one embodiment of the invention.

FIG. 5 shows a schematic drawing of the patient-guided stereoscopic surface imaging and biofeedback system implemented with a treatment system, according to one embodiment of the invention.

### DETAILED DESCRIPTION

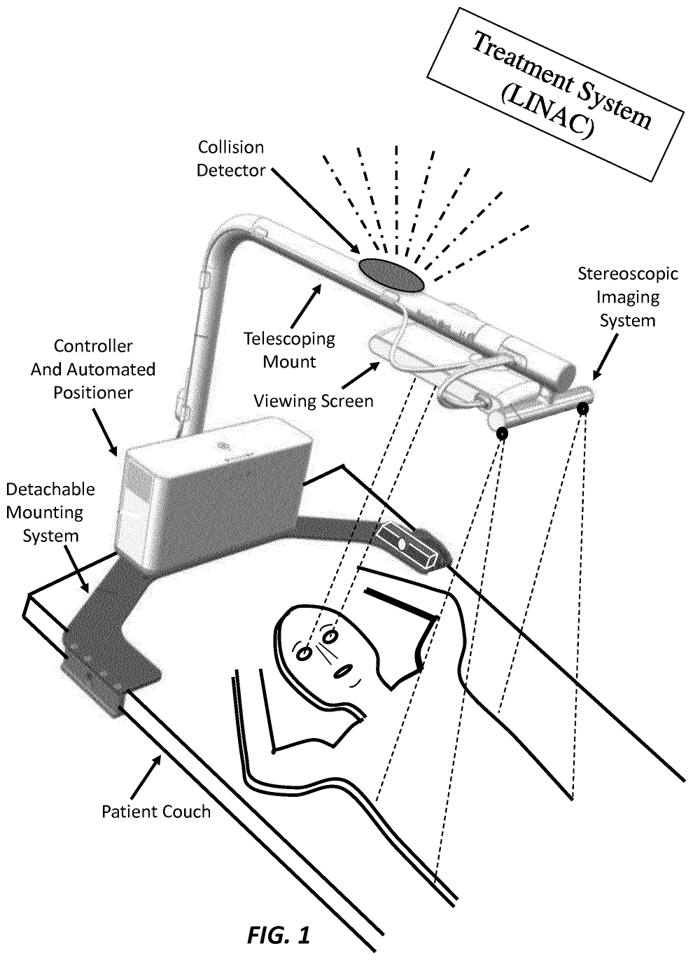

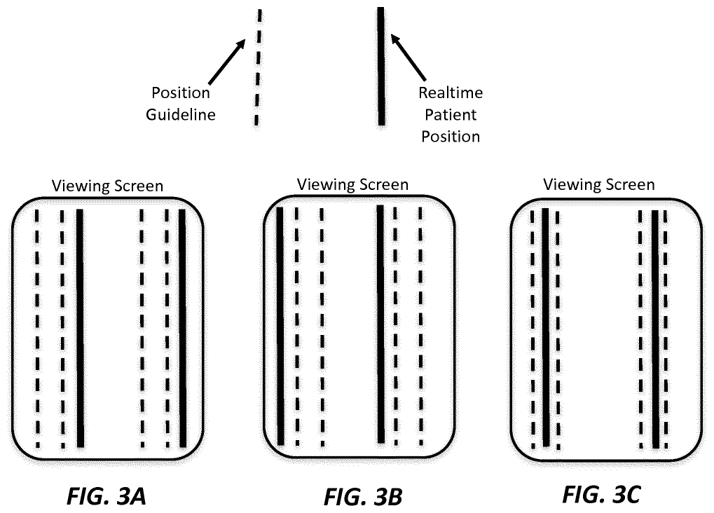

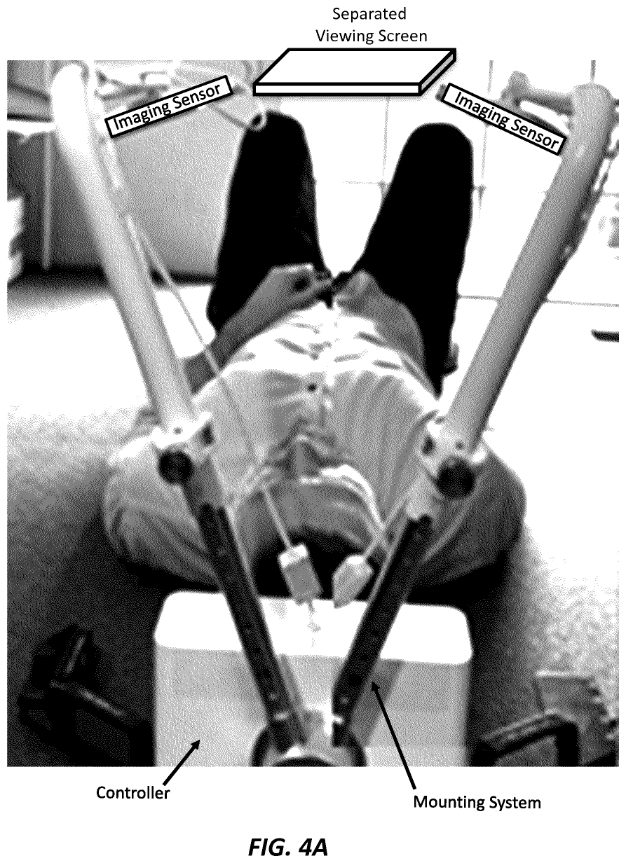

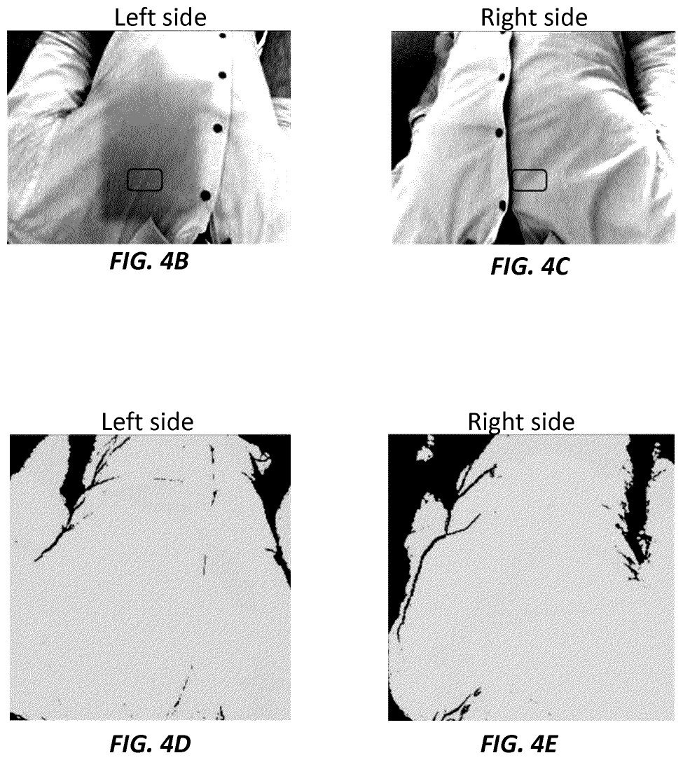

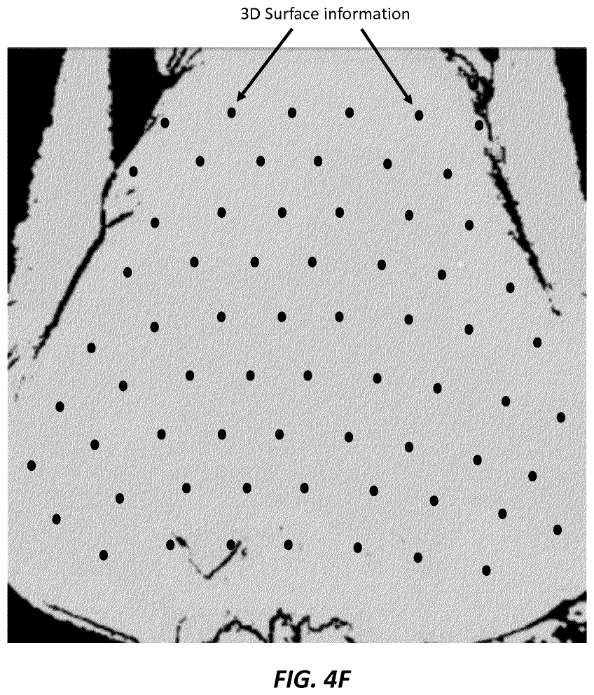

The current invention provides a couch-mounted stereoscopic surface imaging and biofeedback system. FIG. 1 shows a schematic drawing of the patient-guided stereoscopic surface imaging and biofeedback system, according to one embodiment of the invention. Here, a patient couch mounting system is shown holding an array of at least two imaging sensors, and a viewing screen configured to display images from the imaging sensors. A controller is configured to control the imaging sensors and the viewing screen, where the patient couch mounting system is configured to position the imaging sensors for imaging a patient under test on a patient couch from multiple viewing angles. The patient couch mounting system is fixedly attachable to the patient couch, where the viewing screen is disposed in a position that is viewable by the subject under test on the patient couch during the sensor imaging. The imaging sensors, the viewing screen, and the controller are configured to output to a user 3D surface information of the patient under test (see FIG. 4F), extrapolated 2D patient under test position information, and 1D patient under test position information (see FIGS. 4A-4E), where the controller is configured to control the viewing screen to display the images from the imaging sensors, where the viewing screen further displays patient position boundary markers (see FIGS. 3A-3C), where the patient position boundary markers are configured to overlay the displayed images on the viewing screen to provide biofeedback to a patient under test during radiotherapy treatment. FIG. 1 further shows the system can include collision detection for patient safety.

In a further aspect of the invention, the imaging sensors can include a camera, an infra-red imager, or an ultrasound imager, where the sensors are configured to operate independently or simultaneously.

In one aspect of the invention, the imaging sensors are positioned over any region of the patient couch by the patient couch mounting system. Further, the positioning of the imaging sensors or the viewing screen include automated or manual positioning.

According to a further aspect of the invention, the patient couch mounting system is detachably mounted to the patient couch.

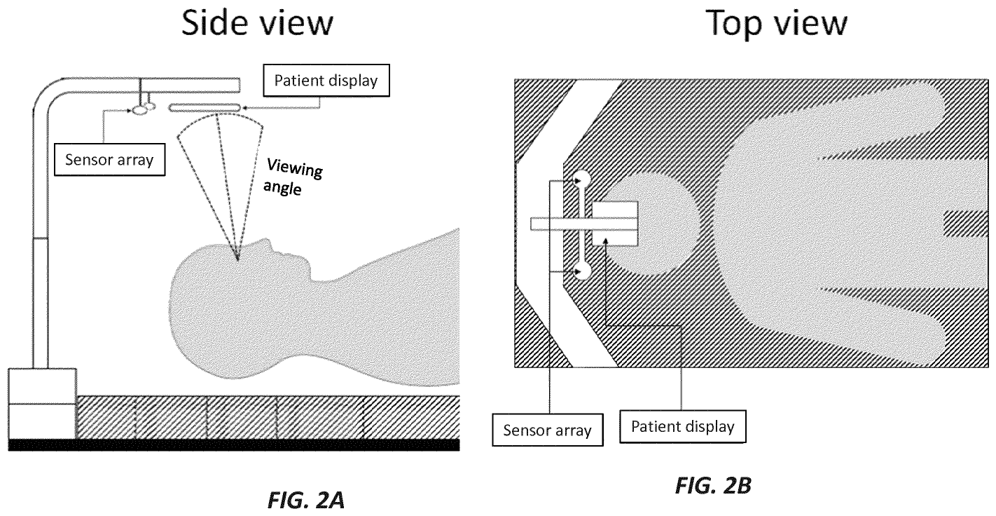

One embodiment of the invention is shown FIGS. 2A-2B. According to the invention, a couch mounted bracket supports an array of two or more sensors that are configured to image the patient from multiple viewing angles, and a feedback viewing screen is disposed to provide real-time feedback to the patient to guide them to an acceptable or ideal position or anatomic or physiologic state, which is useful in a radiotherapy treatment, for example.

In one embodiment, a patient is positioned under the feedback viewing screen showing an ideal position outline on the display. The patient matches their own image to the ideal position outline on the feedback viewing screen to achieve the required patient positioning for treatment without the need for an immobilization device. In one embodiment, the biofeedback system includes a gamified interface, where FIGS. 3A-3C show an augmented reality interface, or a gamified interface and an augmented reality interface for visual biofeedback. As shown the patient guidelines and the real-time patient position with respect to the guidelines to aid the patient to self-adjust their position using the imaging sensors. FIG. 3A shows the patient positioned off-center to the right, FIG. 3B shows the patient positioned off-center to the left, and FIG. 3C shows the patient positioned in the correct center-position. In another aspect of the invention, the imaging sensors and the viewing screen comprise a wireless connectivity, or a wired connectivity. The current invention is compatible to use in patient recognition for assisting with treatment safety, for example facial or vocal recognition of the patient.

FIGS. 2A-2F show an example of stereoscopic surface imaging, where depth and displacement information is taken from each sensor at different angles and reconstructed to form a 3D surface image of the patient surface, combining the depth and displacement information to a single 3D surface map. A 3D surface map is determined by each individual sensor, where each pixel corresponds to a measured position in the 3D space. Because a single camera is observing the subject under test from a single view, the 3D surface map is incomplete, as certain regions of the surface are out of view from the sensor. The invention uses additional sensors to complete the full 3D map of the surface being observed. More specifically, depth maps from multiple cameras can be combined through ray-tracing using the knowledge of the position of each camera in a special domain. According to one embodiment of the invention, combining the depth maps from multiple cameras to create a combined 3D depth maps has several advantages: (1) The overall scene being viewed is larger; (2) Unseen or partially-seen objects from one view may be resolved from another view; and (3) More accurate depth measurements can be obtained when combining the depth information from multiple cameras of the same surface point.

According to the invention, regions can be selected on the 3D surface to extract specific 3D, 2D, or 1D surface position and motion information. FIGS. 4A-4F show an example of real-time stereoscopic surface imaging, where the where 3D surface information (FIG. 4F) is extrapolated from 2D and 1D position information (FIG. 4A-4E), according to one embodiment of the invention.

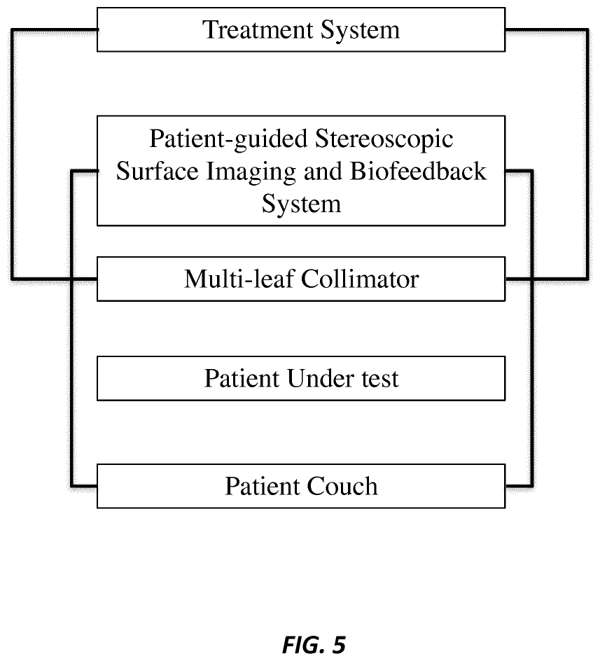

FIG. 5 shows a schematic drawing of the patient-guided stereoscopic surface imaging and biofeedback system implemented with a treatment system, according to one embodiment of the invention. In a further aspect of the invention, the patient-guided surface stereoscopic imaging and biofeedback system is compatible with photon therapy, compatible with proton therapy, compatible with electron therapy, or integrated with a gating interface of cancer therapy system. Further, the system is compatible with the controllable axes of a linear accelerator that includes multileaf collimator positions, couch positions, couch angles, collimator angles, and gantry angles.

In a further aspect of the invention, the patient-guided surface stereoscopic imaging and biofeedback system is MRI compatible.

Mounting the system to the couch itself negates complications arising from the relative motion between the couch and conventional wall/ceiling-mounted devices, in addition to being more proximal to the patient enabling more precise measurements of the patient surface. Providing biofeedback to the patient provides a motion-management system to adapt to the patient's head and body position negating the need for the uncomfortable and potentially hazardous immobilization equipment.

The current invention has treatment applications that include brain, head and neck, breast, lung, thoracic, abdominal and pelvic medical imaging and radiotherapy procedures, including both photon and particle therapy applications.

The present invention has now been described in accordance with several exemplary embodiments, which are intended to be illustrative in all aspects, rather than restrictive. Thus, the present invention is capable of many variations in detailed implementation, which may be derived from the description contained herein by a person of ordinary skill in the art. All such variations are considered to be within the scope and spirit of the present invention as defined by the following claims and their legal equivalents.

What is claimed:

1) A patient-guided surface stereoscopic imaging and biofeedback system, comprising:
a) a patient couch mounting system;
b) an array of at least two imaging sensors;
c) a viewing screen configured to display images derived from said imaging sensors;
d) a controller configured to control said imaging sensors and said viewing screen;
wherein said patient couch mounting system is configured to position said imaging sensors for imaging a patient under test on a patient couch from multiple viewing angles, wherein said patient couch mounting system is fixedly attachable to said patient couch, wherein said viewing screen is disposed in a position that is viewable by said subject under test on said patient couch during said sensor imaging, wherein said controller is configured to control said viewing screen to display said images from said imaging sensors, wherein said imaging sensors, said viewing screen, and said controller are configured to output to a user 3D surface information of said patient under test, extrapolated 2D patient under test position information, and 1D patient under test position information, wherein said viewing screen further displays patient position boundary markers, wherein said patient position boundary markers are configured to overlay said displayed images on said viewing screen to provide biofeedback to a patient under test during radiotherapy treatment.

2) The patient-guided stereoscopic surface imaging and biofeedback system of claim 1, wherein said biofeedback informs said patient under test of a correct position to adjust to and to maintain.

3) The patient-guided stereoscopic surface imaging and biofeedback system of claim 1, wherein said biofeedback system comprises a gamified interface, an augmented reality interface, or a gamified interface and an augmented reality interface for visual biofeedback.

4) The patient-guided stereoscopic surface imaging and biofeedback system of claim 1, wherein said imaging sensors, said viewing screen, and said controller are configured to output to a user 3D surface information of said patient under test, extrapolated 2D patient under test position information, and 1D patient under test position information.

5) The patient-guided stereoscopic surface imaging and biofeedback system of claim 1, wherein said imaging sensors are selected from the group consisting of a camera, an infra-red imager, and an ultrasound imager, wherein said sensors are configured to operate independently or simultaneously.

6) The patient-guided stereoscopic surface imaging and biofeedback system of claim 1, wherein said imaging sensors are connected to said viewing screen, or separated from said viewing screen.

7) The patient-guided stereoscopic surface imaging and biofeedback system of claim 1, wherein said imaging sensors are positioned over any region of said patient couch by said patient couch mounting system.

8) The patient-guided stereoscopic surface imaging and biofeedback system of claim 1, wherein said imaging sensors and said viewing screen comprise a wireless connectivity, or a wired connectivity.

9) The patient-guided stereoscopic surface imaging and biofeedback system of claim 1, wherein said patient couch mounting system is detachably mounted to said patient couch.

10) The patient-guided stereoscopic surface imaging and biofeedback system of claim 1, wherein said patient-guided surface stereoscopic imaging and biofeedback system is integrated with a gating interface of a cancer therapy system.

11) The patient-guided stereoscopic surface imaging and biofeedback system of claim 1, wherein said patient-guided surface stereoscopic imaging and biofeedback system is compatible with photon therapy, or compatible with proton therapy.

12) The patient-guided stereoscopic surface imaging and biofeedback system of claim 1, wherein said patient-guided surface stereoscopic imaging and biofeedback system is compatible with a controllable axes of a linear accelerator selected from the group consisting of multileaf collimator positions, couch positions, couch angles, collimator angles, and gantry angles.

13) The patient-guided stereoscopic surface imaging and biofeedback system of claim 1, wherein said patient-guided surface stereoscopic imaging and biofeedback system is MRI compatible.

14) The patient-guided stereoscopic surface imaging and biofeedback system of claim 1, wherein positioning of said imaging sensors or said viewing screen comprise automated or manual positioning.

15) The patient-guided stereoscopic surface imaging and biofeedback system of claim 1 further comprising collision detection for patient safety.
